(19) United States
(12) Patent Application Publication
(10) Pub. No.: US 2026/0137876 A1
(43) Pub. Date: May 21, 2026

(54) Closed System Injection Device Utilizing a Concentric Needle and Tube

(71) Applicant: Becton, Dickinson and Company, Franklin Lakes, NJ (US)

(72) Inventors: Narsi Reddy Sanikommu, Kanigiri (IN); Aman Desai, Bengaluru (IN); Kowshika K, Tiruppur (IN); Jason Andrew Wine, Brea, CA (US)

(21) Appl. No.: 18/952,300

(22) Filed: Nov. 19, 2024

(51) Int. Cl. A61M 5/32

(52) U.S. Cl. CPC A61M 5/321

(57) ABSTRACT

An injection device is disclosed. The injection device includes a device body including first passage therein and a port defining a lumen in fluid communication with the first passage. The injection device includes a tube coupled to the device body, the tube defining a proximal region, a distal region, and a bore in fluid communication with the first passage and lumen, and a needle at least partially disposed within the tube, wherein the needle is movable with respect to the tube between a first position in which the needle prevents fluid flow through the distal region of the tube and a second position in which the needle permits fluid flow through the distal region of the tube.

### BACKGROUND OF THE INVENTION

### FIELD OF THE INVENTION

The present disclosure relates generally to devices and methods of use thereof for the closed transfer and/or injection of fluids.

### DESCRIPTION OF RELATED ART

Reconstituting, transporting, and administering hazardous drugs, such as cancer treatments, can put health care providers at risk of exposure to these medications and present a major hazard in the health care environment. For example, nurses treating cancer patients risk being exposed to chemotherapy drugs and their toxic effects when administering these potentially harmful drugs due to insecure connections or inadvertent disconnection of the delivery devices or adapters used to transport or administer such drugs. Unintentional chemotherapy exposure can affect the nervous system, impair the reproductive system, and bring an increased risk of developing blood cancers in the future. In order to reduce the risk of health care providers being exposed to toxic drugs, the secure, closed transfer of these drugs is important. For example, injection of a compound into a container, fluid system, and/or patient typically leaves a residual amount of the compound in the hollow lumen of the injection cannula. When the cannula is removed from the injection site after use, this residual amount of compound may leak or drop onto a surface surrounding the injection site (including, for example, a patient’s skin, a healthcare worker’s hand, and/or a surface on a device or container involved in the injection), which carries the toxicity risks discuses above.

The present disclosure provides systems, devices, and methods of use thereof for injection that reduce or eliminate undesired exposure to such compounds.

### SUMMARY OF THE INVENTION

In accordance with one aspect, the present disclosure is directed to an injection device including a device body including a first passage therein and a port defining a lumen in fluid communication with the first passage, a tube coupled to the device body, the tube defining a proximal region, a distal region, and a bore in fluid communication with the first passage and lume, and a needle at least partially disposed within the tube. The needle being movable with respect to the tube between a first position in which the needle prevents fluid flow through the distal region of the tube and a second position in which the needle permits fluid flow through the distal region of the tube.

In certain configurations, the needle has distal region with a continuous surface devoid of openings. In the first position, a portion of the needle extends distally out of the distal region of the tube, and in the second position, a distal end of the needle is enclosed within the tube. In certain configurations, the needle is displaced axially with respect to the tube to move from the first position to the second position. In other configurations, the needle is axially movable with respect to the device body.

In certain configurations, the injection device further includes an actuator coupled to the needle to move the needle from the first position to the second position. In certain configurations, the actuator is rotatably coupled to the device body. In other configurations, the injection device further includes a connector coupled to the port, the connector including a second passage in fluid communication with the lumen. In certain configurations, the proximal region of the tube is axially affixed to the device body. Optionally, the injection device also includes a septum disposed in the first passage.

In certain configurations, the injection device further includes a sheath attached to the device body and coaxial with the needle, wherein the sheath is axially movable with respect to the needle.

In accordance with another embodiment of the present application, an injection device includes a device body having a first end, a second end, a first passage therein, and a port defining a lumen in fluid communication with the first passage. The injection device further includes a tube defining a proximal region coupled to the second end of the device body, a distal region, and a bore in fluid communication with the first passage and lumen, and a needle at least partially disposed within the tube and axially movable with respect to the tube. The injection device also includes an actuator coupled to the first end of the device body and configured to axially displace the needle with respect to the tube.

In certain configurations, the lumen of the port is angled with respect to the first passage. In other configurations, the injection device further includes a connector coupled to the port, with the connector including a second passage in fluid communication with the lumen. In other configurations, the needle is axially movable from a first position in which a portion of the needle extends distally out of the distal region of the tube, to a second position in which a distal end of the needle is enclosed within the tube. In still other configurations, when the injection device is in the first position, the needle seals the distal region of the tube.

In accordance with another embodiment of the present invention, a method of delivering a fluid includes a piercing a portion of a fluid recipient with a needle of an injection device, the injection device including a device body including a passage therein and a port defining a lumen in fluid communication with the passage. The injection device further including a tube coupled to the device body, the tube including a distal region and a bore in fluid communication with the first passage and lumen, wherein the needle is at least partially disposed within the tube. The method also includes axially moving the needle with respect to the tube and delivering the fluid through the passage and out of the distal region of the tube.

In certain configurations, the step of axially moving the needle with respect to the tube includes moving the needle from a first position in which a portion of the needle extends distally out of the distal region of the tube, to a second position in which a distal end of the needle is enclosed within the tube. In other configurations, in the first position, the needle seals the distal region of the tube. In other configurations, the fluid recipient is at least one of a container or a device.

### BRIEF DESCRIPTION OF THE DRAWINGS

The above-mentioned and other features and advantages of this disclosure, and the manner of attaining them, will become more apparent and the disclosure itself will be better understood by reference to the following descriptions of embodiments of the disclosure taken in conjunction with the accompanying drawings, wherein:

FIG. 1 is a perspective view of an example of an injection device constructed in accordance with the principles of the present invention.

FIG. 2 is a side view of the injection device of FIG. 1.

FIG. 3 is a side cross-sectional view of the injection device of FIG. 1.

FIG. 4 is an exploded assembly view of the injection device of FIG. 1.

FIG. 5 is a side cross-sectional view of an example of an injection device constructed in accordance with the principles of the present invention.

FIG. 6 is a side cross-sectional views of an example of an injection device with an adjustable sheath constructed in accordance with the principles of the present invention.

FIG. 7 is a side cross-sectional views of an example of an injection device with an adjustable sheath constructed in accordance with the principles of the present invention.

FIG. 8 is a side cross-sectional view of an example of an injection device constructed in accordance with the principles of the present invention.

FIG. 9 is a side cross-sectional view of an example of an injection device constructed in accordance with the principles of the present invention.

FIG. 10 is a side cross-sectional view of examples of configurations for use of an injection device constructed in accordance with the principles of the present invention.

FIG. 11 is a side cross-sectional view of examples of configurations for use of an injection device constructed in accordance with the principles of the present invention.

FIG. 12 is a side cross-sectional views of an example of an injection device constructed in accordance with the principles of the present invention.

FIG. 13 is a side cross-sectional views of an example of an injection device constructed in accordance with the principles of the present invention.

### DETAILED DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the concept as it is oriented in the drawing figures. However, it is to be understood that the concept may assume various alternative variations, except where expressly specified to the contrary. It is also to be understood that the specific devices illustrated in the attached drawings, and described in the following specification, are simply exemplary embodiments of the concept. Hence, specific dimensions and other physical characteristics related to the embodiments disclosed herein are not to be considered as limiting.

The word “comprising” and “comprises”, and the like, does not exclude the presence of elements or steps other than those listed in any claim or the specification as a whole. In the present specification, “comprises” means “includes” and “comprising” means “including.”

As used herein, “at least one of” is synonymous with “one or more of.” For example, the phrase “at least one of A, B, or C” means any one of A, B, or C, or any combination of any two or more of A, B, or C. For example, “at least one of A, B, and C” includes A alone; or B alone; or C alone; or A and B; or A and C; or B and C; or all of A, B, and C.

The term “at least” is synonymous with “greater than or equal to.” The terms “first”, “second”, and the like are not intended to refer to any particular order or chronology, but refer to different conditions, properties, or elements. As used herein, the singular form of “a”, “an”, and “the” include plural referents unless the context clearly dictates otherwise.

All numbers used in the specification and claims are to be understood as being modified in all instances by the term “about”. By “about” is meant within plus or minus twenty-five percent of the stated value. However, this should not be considered as limiting to any analysis of the values under the doctrine of equivalents.

Unless otherwise indicated, all ranges or ratios disclosed herein are to be understood to encompass the beginning and ending values and any and all subranges or subratios subsumed therein. For example, a stated range or ratio of “1 to 10” should be considered to include any and all subranges or subratios between (and inclusive of) the minimum value of 1 and the maximum value of 10; that is, all subranges or subratios beginning with a minimum value of 1 or more and ending with a maximum value of 10 or less. The ranges and/or ratios disclosed herein represent the average values over the specified range and/or ratio.

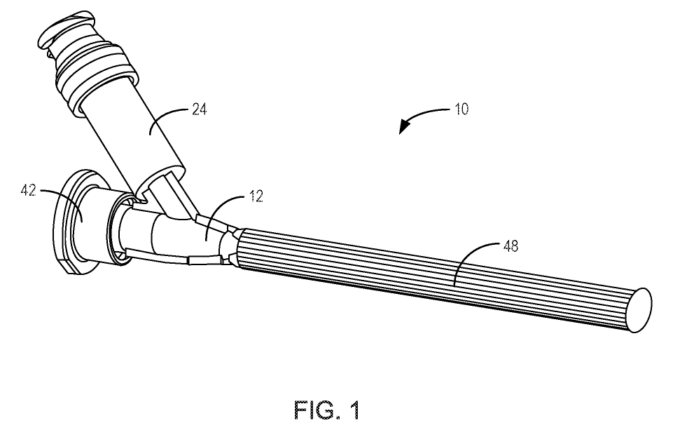

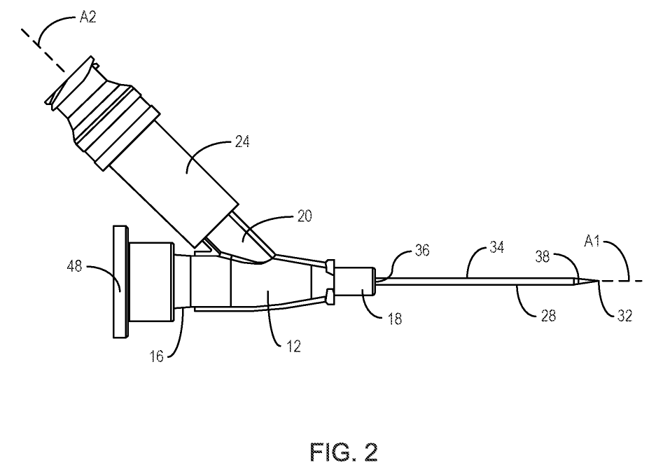

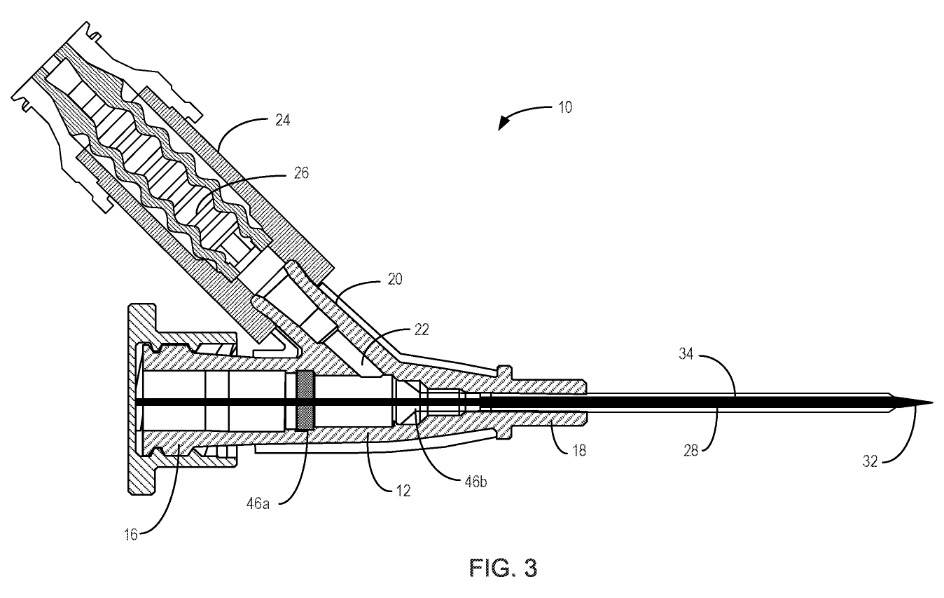

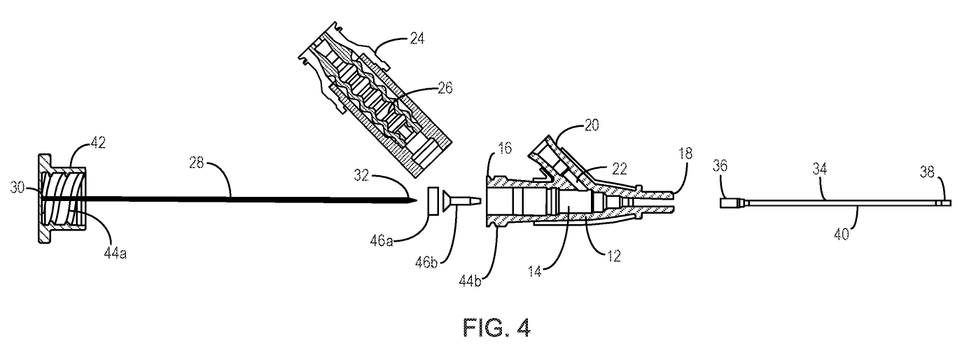

Now referring to FIGS. 1-5, an example of an injection device 10 is shown. The injection device 10 may be configured and/or operable to facilitate the injection of one or more fluids or compounds while reducing the likelihood of inadvertent or unwanted exposure to, or waste of, the fluids or compounds being injected. The injection device 10 may generally include or define a device body 12 defining an interior passage 14 extending from a first end 16 of the device body 12 to a second end 18 of the device body 12. The device body 12 may include or define a port 20 defining or including a lumen 22 extending into or otherwise in fluid communication with the interior passage 14. The device body 12 may include or define a longitudinal axis A1 extending from the first end 16 to the second end 18, and a secondary axis A2 that may extend along the length of the port 20 and/or lumen 22 at a non-parallel angle with respect to the axis A1.

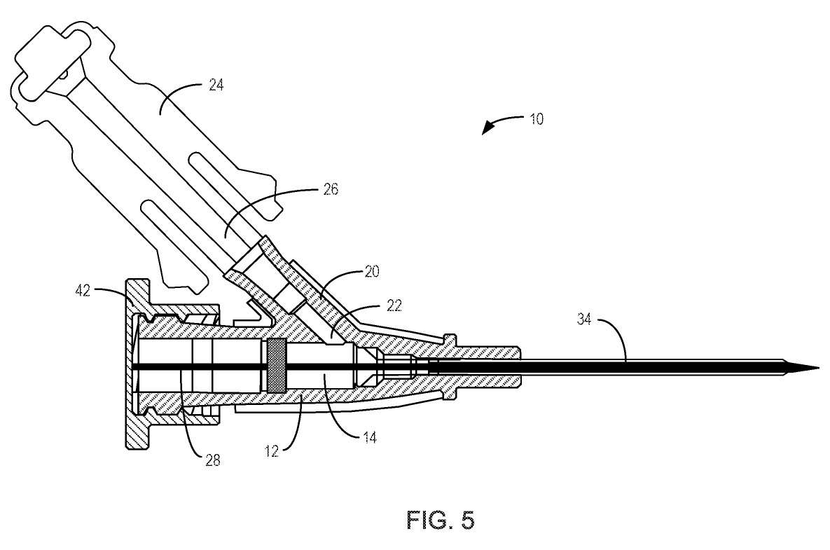

The injection device 10 may include or define a connector 24 coupled to, extending from, or otherwise in fluid communication with the port 20 of the device body 12. The connector 24 may include or define a passage 26 therein connected to and/or in fluid communication with the lumen 22 of the port 20 and the passage 14 of the device body 12. The connector 24 may provide one or more matable interfaces or connection features to enable or facilitate attaching a fluid source to the injection device 10 (such as, e.g., syringes, closed-system transfer devices, intravenous delivery system components, pumps, fluid containers, or the like). For example, the connector 24 may include or define a luer lock connection (e.g., as shown in FIGS. 1-4), one or more threaded surfaces, one or more needleless connections, a re-sealable stopper or other pierceable layer, a closed system drug device interface (e.g., such as the PhaSeal™ Optima System offered by Becton Dickinson and Company, as shown in FIG. 5), or otherwise. The connector 24 may be releasably engageable with the port 20 or integral with the port 20 and device body 12.

The injection device 10 may include a needle 28 coupled to the device body 12 and configured or operable to pierce or penetrate a desired injection site or recipient (e.g., including but not limited to a patient, a container, a device, or other desired fluid destination). The needle 28 may include or define a proximal region 30 and a distal region 32. The distal region 32 may include or define a substantially conical, tapered, and/or sharp tip sufficient to penetrate or pierce an injection location. The needle 28 may include or define a substantially solid and sealed outer surface on or about the distal region 32 and/or may be constructed as a solid member devoid of openings or lumens such that the needle 28 does not provide any fluid port or fluid communication therethrough. The needle 28 may include or define a plurality of different diameters along at least a portion of a length thereof. For example, the distal region 32 of the needle 28 may include or define a larger diameter compared to a diameter of the needle located proximal of the distal region 32.

The needle 28 may be at least partially disposed within the passage 14 of the device body 12. The needle 28 may be substantially aligned with and/or parallel to the axis A1, and in some non-limiting aspects, may extend outward from the second end 18 such that the distal region 32 of the needle 28 is external to the device body 12. The needle 28 may be coupled to the device body 18 such that the needle 28 is movable with respect to the device body 12. The needle 28 may be movable with respect to the device body 12 in an axial direction substantially parallel to and/or along the axis A1.

The injection device 10 may include a tube 34 coupled to and extending from the first end 16 of the device body 12. The tube 34 may define a proximal region 36, a distal region 38, and an internal bore 40 extending between the proximal and distal regions 36, 38 and in fluid communication with the passage 14 of the device body, the lumen 22 of the port 20, and/or the passage 26 of the connector 24. The tube 34 may be coaxially aligned with and/or concentrically positioned with the needle 28 such that at least a portion of the needle 28 is positioned within the bore 40 of the tube 34. The tube 34 may be axially-affixed and/or immovable with respect to the device body 12, while the needle 38 is movable with respect to the tube 34 and the device body 12. The needle 28 may be movable with respect to the tube 34 in an axial direction substantially parallel to and/or along the axis A1.

The tube 34 and the needle 28 may be comparatively sized and/or shaped to define a fluid flow path within and along the at least a portion of the bore 40 with the needle 28 positioned therein. For example, the bore 40 may define a diameter larger than an outer diameter of the needle 28 along concentric sections thereof such that a fluid may be injected or delivered through a space created or existing between the outer surface of the needle 28 and the sidewall or inner surface of the tube 34 defining the bore 40. The tube 34 and the needle 28 may be comparatively sized and/or shaped such that the distal region 32 of the needle 28 is configured or operable to substantially obstruct or seal the bore 40 in or about the distal region 38 of the tube 34. For example, the distal region 38 of the tube 34 and bore 40 may have a tapered or reduce diameter than one or more proximal sections or regions of the tube 34 and bore 40 such that the distal region 38 of the tube 34 abuts and seals against the outer surface of the distal region 32 of the needle 28.

The injection device 10 may include or define an actuator 42 configured or operable to adjust or select a relative position between the needle 28 and the tube 34. In the illustrated example, the actuator 42 may be configured or operable to adjust an axial position of the needle 28 substantially parallel to or otherwise along the axis A1 with respect to the tube 34, which may be axially static or immovably affixed to the device body 12. In another example (not shown), the tube 34 may be movable with respect to the needle 28, which may be axially static or immovably affixed to the device body 12.

The actuator 42 may be coupled to and/or movable with respect to the device body 12 to facilitate the movement and features described herein. For example, the actuator 42 may include or define a dial or knob-like structure coupled to the first end 16 of the device body. The actuator 42 may include or define a threaded surface 44a engaged with or coupled to a threaded surface 44b on the first end 16 of the device body 12. The needle 28 may be axially affixed to the actuator 42 such that, when the actuator 42 is rotated, the threaded engagement axially displaces the actuator 42 with respect to the device body 12, thus axially displacing the needle 28 with respect to the device body and/or the tube 34.

In another example, the needle 28 may be threadably engaged to the actuator 42 such that rotation of the actuator 42 axially displaces the needle 28 with respect to the device body 12 and/or the tube 34 while the actuator 42 remains axially affixed or static with respect to the device body 12. In another example, the actuator 42 may be operable or configured to axially displace the tube 34 with respect to the needle 28 and/or the device body 12 to provide the features and functions disclosed herein.

The injection device 10 may include one or more seals, barriers, valves, or other protective mechanisms preventing fluid flow, or providing selective fluid flow, through one or more portions of the device 10. For example, a septum 46a may be disposed within the passage 14 of the device body 12 to prevent fluid flow from reaching the actuator 42 and/or first end 16 of the device body 12 during use. Another gasket or seal 46b may be disposed between a junction or interface between the proximal region 36 of the tube 34 and an interior surface or coupling with the second end 18 of the device body 12 to prevent leaks. One or more seals, barriers, valves, or other fluid control mechanisms may be positioned on, in, or about the passage 14 of the device body 12, the lumen 22 of the port 20, the passage 26 of the connector 24, and/or the bore 40 of the tube 34.

The injection device 10 may include a cap 48 releasably engageable with the device body 12 and/or otherwise configured to enclose or protect at least a portion of the needle 28. The cap 48 may be releasably engageable with the second end 18 of the device body 12 and have sufficient size and shape to enclose the needle 48 and/or tube 34 therein during transport or storage prior to use of the injection device 10.

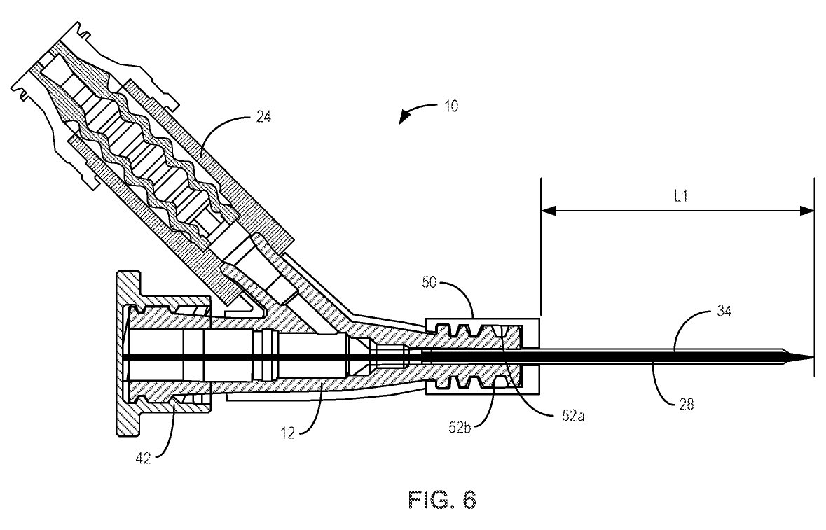

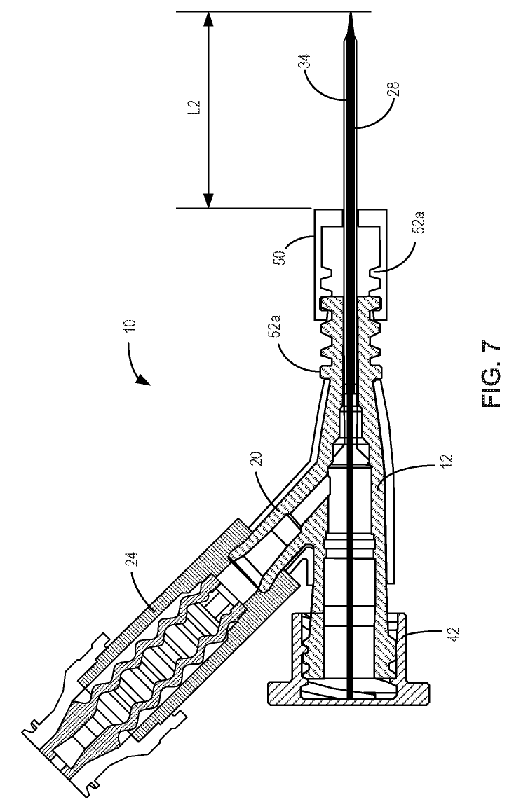

Now referring to FIGS. 6 and 7, the injection device 10 may include or define a sheath 50 having an adjustable position and/or length operable or configured to limit and/or pre-select a penetration depth of the needle 28 and/or tube 34. For example, the sheath 50 may be movable or extendable axially with respect to the second end 18 of the device body in a direction substantially parallel to or otherwise along the axis A1. The sheath 50 may be movable or extendable from a first position, as shown in FIG. 6, with an exposed length L1 of the needle 28 extending distally with respect to the sheath 50, to a second position, as shown in FIG. 7, with a reduced exposed length L2 of the needle 28 extending distally with respect to the sheath 50.

The sheath 50 may be coaxially and/or concentrically mounted or positioned with respect to the needle 28 and/or the tube 34. The sheath 50 may be movable and/or extendable into a plurality of discrete axial positions at preselected locations to provide a predetermined length of exposed needle 28 for subsequent injection. For example, the sheath 50 may be movable or extendable in 1mm increments to select a penetration depth of the needle 28. Alternatively, the sheath 50 may be movable or extendable along a continuous range to provide a desired exposed length of the needle 28.

The sheath 50 may include various mechanism to enable the axial movement and/or extension described herein. For example, the sheath 50 may include a threaded surface 52a engaged to a threaded surface 52b of the device body 12 that enables axial displacement of the sheath 50 relative to the device body 12, and thus the needle 28. In other examples, the sheath 50 may include or define a bellows structure, may be constructed from an elastically deformable or stretchable material, and/or may include other mechanisms as known in the art.

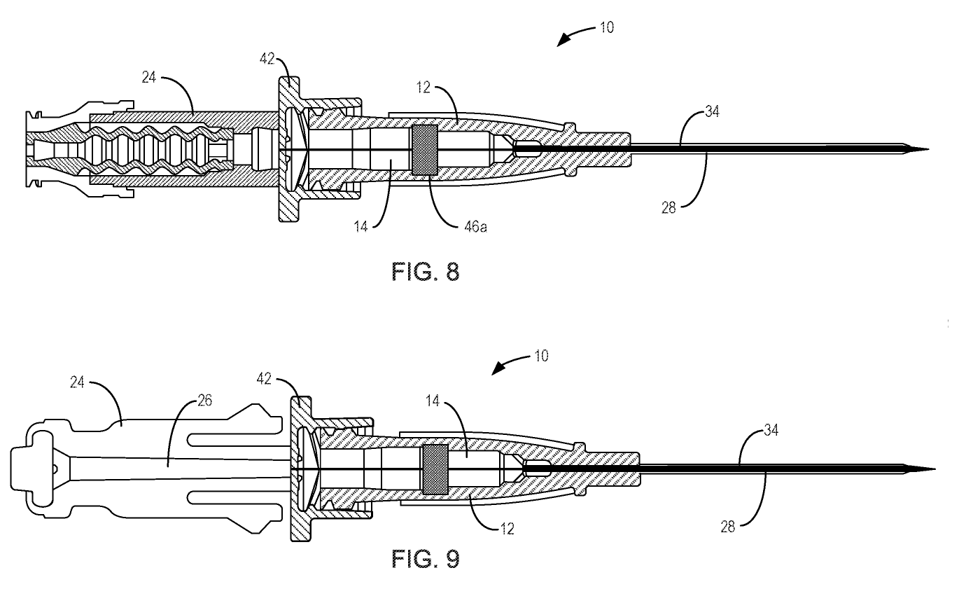

Now referring to FIGS. 8 and 9, the injection device 10 may have a substantially linear configuration as an alternative to the angled port configuration illustrated in FIGS. 1-7. The port 20 and/or connector 24 may be coupled to, integrated with, or otherwise extend from the first end 16 of the device body 12. The connector 24 may include a luer-type connector, as shown in FIG. 8 and described herein, a closed system drug device interface as shown in FIG. 9 and described herein, or any other suitable connection type. The port 20 and/or connector 24 may be coaxially and/or concentrically mounted on or positioned with respect to the passage 14, actuator 42, needle 28, and/or tube 34 to provide the features and functions disclosed herein with respect to each of those components. The port 20 may, for example, constitute an opening or access integral with the actuator 42. In another example, the passage 26 of the connector 24 may couple to and/or feed directly into the passage 14 of the device body 12.

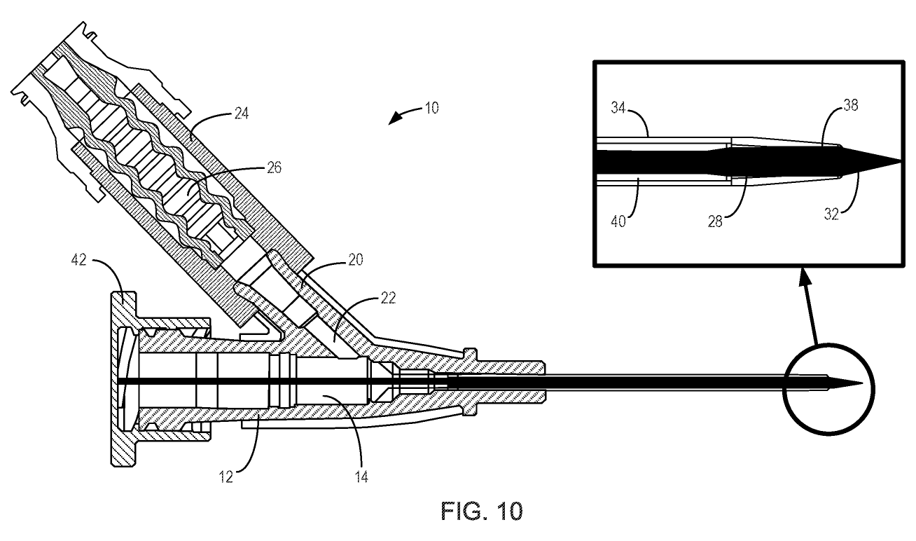

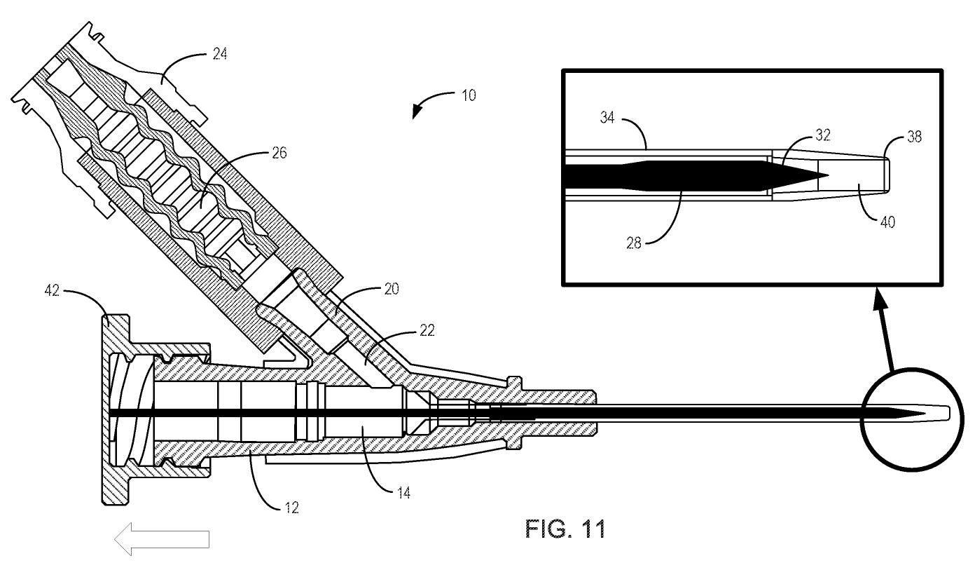

Now referring to FIGS. 10 and 11, exemplary configurations for use of the injection device 10 are illustrated. For example, the injection device 10 may be in a first configuration or position such that the needle 28 is axially aligned or positioned to substantially seal or close the bore 40 of the tube 34 at the distal region 38 of the tube 34, as shown in FIG. 10. A fluid source may be coupled to the connector 24 containing fluid to be delivered by and through the injection device 10. In this first configuration or position, the passage 14 of the device body 12, the lumen 22 of the port 20, and the passage 26 of the connector 24 may be in fluid communication with the space between the bore 40 and the needle 28 within the tube 34, but there is no fluid exit or flow path from the distal region 38 of the tube 34. In this first configuration or position, at least a portion of the distal region 32 of the needle 28 may be axially extending from and beyond the distal region 38 of the tube 34. In this first configuration or position, the needle 28 may be directed towards an insertion site or target, and used to pierce or penetrate the injection target (whether a patient, container, or the like as described herein). In one example, the needle 28 may perform a subcutaneous injection to position both the distal region 32 of the needle 28 and the distal region 38 of the tube 34 into and beneath a skin surface. In another example, the needle 28 may be used to penetrate or pierce a layer, cover, stopper, or the like of a container, fluid system, or other medical device to deliver or transfer fluids.

Once the needle 28 and the tube 34 have been adequately positioned to a desired depth at least partially through and/or within the injection site or target, the relative positioning between the needle 28 and the tube 34 may be adjusted to allow fluid to exit from the bore 40 and through the distal region 38 of the tube, as shown in FIG. 11. For example, the actuator 42 may be operated to axially retract or displace the needle towards the device body 12, thereby opening the bore 40 to fluid communication with the surrounding injection site or target. Fluid then may be delivered through a fluid pathway including the passage 26 of the connector 24, the lumen 22 of the port 20, through the passage 14 of the device body 12, and the bore 40 of the tube 34.

Once the desired amount of fluid has been injected or dispensed through the bore 40, the needle 28 may be re-positioned to again seal the distal region 38 of the tube 34 (e.g., to the first position as shown in FIG. 10). The needle 28 and tube 34 may then be retracted or withdrawn from the injection site without the risk of any residual fluid exiting the distal region 38 of the tube 34, and thus the injection device 10.

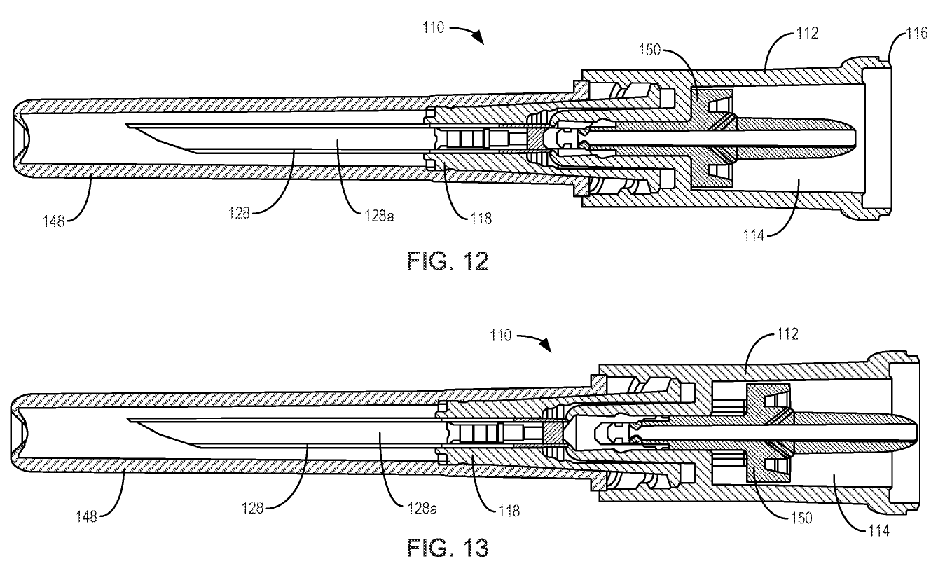

Now referring to FIGS. 12 and 13, another example of an injection device 110 is shown. The injection device 10 includes a substantially linear fluid flow configuration similar to the examples illustrated and described with respect to FIGS. 8 and 9. The injection device 110 may include features and components similar to those presented and described with respect to the injection device 10 as disclosed herein. The injection device 110 may generally include or define a device body 112 with a passage 114 therein extending from a first end 116 of the injection device 110 to a second end 18 of the device 110. A needle 128 may be coupled to the second end of the device body 112, and the needle 128 may include or define a lumen or flowpath 128a therein. The injection device 110 may include a cap 148 coupled to an exterior of the device body to enclose the needle 128 during transport or storage prior to use. The injection device 110 may include a plug 150 axially movable within at least portion of the passage 114 to selectively permit or prevent fluid communication between the passage 114 and the lumen 128a of the needle 128. The plug 150 may be movable axially with respect to the device body 112 in from a first position where the plug 150 prevents fluid flow to and through the needle 128 (as shown in FIG. 12), to a second position where the plug 150 permits fluid flow to and through the needle 128 (as shown in FIG. 13 ).

In an example of use, the injection device 110 may be coupled to a fluid source at the first end 116 either directly or through an intermediate connector (not shown). The needle 128 may be used to pierce or penetrate an injection or fluid transfer target as described herein, and the plug 150 may be manipulated or actuated to modulate, permit, and/or prevent fluid flow from the passage 114 of the device body 112 through the needle 128 and to the injection target.

It will be appreciated by persons skilled in the art that the present disclosure is not limited to what has been particularly shown and described herein above. In addition, unless mention was made above to the contrary, it should be noted that all of the accompanying drawings are not to scale. Of note, the system components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein. Moreover, while certain embodiments or figures described herein may illustrate features not expressly indicated on other figures or embodiments, it is understood that the features and components of the examples disclosed herein are not necessarily exclusive of each other and may be included in a variety of different combinations or configurations without departing from the scope and spirit of the disclosure. A variety of modifications and variations are possible in light of the above teachings without departing from the scope and spirit of the disclosure, which is limited only by the following claims.

What is claimed is:

1. An injection device, comprising:
a device body including a first passage therein and a port defining a lumen in fluid communication with the first passage;
a tube coupled to the device body, the tube defining a proximal region, a distal region, and a bore in fluid communication with the first passage and lumen; and
a needle at least partially disposed within the tube, wherein the needle is movable with respect to the tube between a first position in which the needle prevents fluid flow through the distal region of the tube and a second position in which the needle permits fluid flow through the distal region of the tube.

2. The injection device of claim 1, wherein the needle has distal region with a continuous surface devoid of openings.

3. The injection device of claim 1, wherein in the first position, a portion of the needle extends distally out of the distal region of the tube, and in the second position, a distal end of the needle is enclosed within the tube.

4. The injection device of claim 1, wherein the needle is displaced axially with respect to the tube to move from the first position to the second position.

5. The injection device of claim 1, wherein the needle is axially movable with respect to the device body.

6. The injection device of claim 1, further comprising an actuator coupled to the needle to move the needle from the first position to the second position.

7. The injection device of claim 6, wherein the actuator is rotatably coupled to the device body.

8. The injection device of claim 1, further comprising a connector coupled to the port, the connector including a second passage in fluid communication with the lumen.

9. The injection device of claim 1, wherein the proximal region of the tube is axially affixed to the device body.

10. The injection device of claim 1, further comprising a septum disposed in the first passage.

11. The injection device of claim 1, further comprising a sheath attached to the device body and coaxial with the needle, wherein the sheath is axially movable with respect to the needle.

12. An injection device, comprising:
a device body including:
a first end,
a second end,
a first passage therein, and
a port defining a lumen in fluid communication with the first passage;
a tube defining a proximal region coupled to the second end of the device body, a distal region, and a bore in fluid communication with the first passage and lumen;
a needle at least partially disposed within the tube and axially movable with respect to the tube; and
an actuator coupled to the first end of the device body and configured to axially displace the needle with respect to the tube.

13. The injection device of claim 12, wherein the lumen of the port is angled with respect to the first passage.

14. The injection device of claim 12, further comprising a connector coupled to the port, the connector including a second passage in fluid communication with the lumen.

15. The injection device of claim 12, wherein the needle is axially movable from a first position in which a portion of the needle extends distally out of the distal region of the tube, to a second position in which a distal end of the needle is enclosed within the tube.

16. The injection device of claim 15, wherein in the first position, the needle seals the distal region of the tube.

17. A method of delivering a fluid, comprising:
piercing a portion of a fluid recipient with a needle of an injection device, the injection device comprising:
a device body including a passage therein and a port defining a lumen in fluid communication with the passage,
a tube coupled to the device body, the tube including a distal region and a bore in fluid communication with the first passage and lumen, wherein the needle is at least partially disposed within the tube;
axially moving the needle with respect to the tube; and
delivering the fluid through the passage and out of the distal region of the tube.

18. The method of claim 17, wherein axially moving the needle with respect to the tube includes moving the needle from a first position in which a portion of the needle extends distally out of the distal region of the tube, to a second position in which a distal end of the needle is enclosed within the tube.

19. The method of claim 18, wherein in the first position, the needle seals the distal region of the tube.

20. The method of claim 17, wherein the fluid recipient is at least one of a container or a device.
